(19) United States
(12) Patent Application Publication
(10) Pub. No.: US 2010/0216240 A1
(43) Pub. Date: Aug. 26, 2010

(54) NON-INVASIVE AUTOMATED CELL PROLIFERATION APPARATUS

(75) Inventors: Francis Sean Moolman, Pretoria (ZA); Kersch Naidoo, Pretoria (ZA); Adriaan Jacobus Van Wyk, Pretoria (ZA)

Correspondence Address: BARNES & THORNBURG LLP, 750-17TH STREET NW, SUITE 900, WASHINGTON, DC, 20006-4675, US

(73) Assignee: CSIR, Brummeria, Pretoria (ZA)

(21) Appl. No.: 12/678,450

(22) PCT Filed: Sep. 5, 2008
(86) PCT No.: PCT/IB2008/053604

§ 371 Date: Mar. 16, 2010

(30) Foreign Application Priority Data
Sep. 7, 2007 (ZA) 2007/07720

(51) Int. Cl. C12N 5/02; C12M 1/00; C12N 1/00

(52) U.S. Cl. 435/395; 435/289.1; 435/243

(57) ABSTRACT

A cell proliferation apparatus for the automated culturing of cells, the proliferation apparatus including a bioreactor having contained therein a stimulus-responsive three dimensional (3D) cell scaffold, the stimulus-responsive three-dimensional (3D) cell scaffold being operable reversibly to change its surface properties between hydrophilic and hydrophobic states. The cell scaffold comprises a surface layer of thermo-responsive polymer.

### FIELD OF THE INVENTION

THIS INVENTION relates to cell culture technology. More particularly, the invention relates to a cell proliferation apparatus, and a method of culturing cells in a non-invasive, continuous manner.

### BACKGROUND OF THE INVENTION

Conventional in vitro cell culturing of anchorage-dependant cells has certain inherent limitations that have hampered progress in many fields, including cell, tissue, and genetic engineering. Traditionally, anchorage-dependent cells have been cultured on flat two-dimensional (2D) polystyrene culture dishes. Cells are removed from the surfaces of such dishes either via proteolytic enzymatic digestion or mechanical methods, once a monolayer of cells has formed. When large cell quantities are required, as is the case for tissue and genetic engineering, the process of dividing cells, seeding, cell growth until confluency and subsequent removal from the polystyrene dishes is repeated until the required number of cells is obtained. Conventional monolayer cell culturing is cumbersome, highly time consuming and is labour-intensive, which increases the risk of cell culture contamination at every harvesting or culture splitting event.

Two-dimensional (2D) cultures typically do not mimic in vivo tissues as well as so-called 3D cultures, especially with regard to cell shape and cellular environment. For in vitro cell culturing, the ideal cell scaffold should display a three-dimensional (3D) morphology similar to the physiological extracellular matrix (ECM). Three-dimensional systems exhibit a much closer approximation to the cell microenvironment in vivo because of improved cell-cell interaction and nutrient, oxygen and waste exchange, augmenting cell viability and function.

The harsh enzymatic or mechanical detachment methods to release adherent cells in 2D and 3D cell culture have been shown to adversely affect cell morphology and function. Enzymatic digestion, typically using trypsin, has been shown to damage the extracellular matrix (ECM) of cultured cells, producing cells that are disaggregated and rounded. Additionally, cell-cell junction proteins as well as receptor proteins present on the cell membrane are frequently damaged. Mechanical release methods produce cells which are surrounded by a crystalline matrix with a compromised ECM. Damage to the ECM is known to lead to a loss of cellular activity and function, resulting in impaired cell growth and differentiation.

The present invention is aimed at addressing certain of the above issues.

### SUMMARY OF THE INVENTION

According to one aspect of the invention, there is provided a cell proliferation apparatus for the automated culturing of cells, the proliferation apparatus including a bioreactor having contained therein a stimulus-responsive three dimensional (3D) cell scaffold.

The stimulus-responsive three-dimensional (3D) cell scaffold may reversibly change its surface properties between hydrophilic and hydrophobic states.

The scaffold material may be defined by a matrix selected from any one or more of fibres, semi-permeable or non-permeable hollow fibres, hydrogels, particles and monolithic porous scaffolds made from either polymers or ceramics. The scaffold may comprise a semi-permeable hollow fibre matrix.

The scaffold may be selected from any one of polystyrene, polypropylene, polyethylene, polyesters, polyamides, natural polymers (such as collagen, hyaluronic acid, and the like) and any other scaffold materials suitable for cell culture.

The scaffold may be modified with a surface layer of thermo-responsive polymer by grafting (i.e. chemical modification).

The grafting technique may be selected from any one or more of: solution free radical polymerisation; gamma radiation; plasma radiation; electron beam radiation; and ultra-violet radiation.

The scaffold may be modified with a surface layer of thermo-responsive polymer by adsorption or physical attachment techniques. The thermo-responsive polymer may be selected from any one or more of poly N-substituted acrylamide, polyethylene-oxide and their respective copolymers, and the like.

The thermo-responsive polymer may be poly-N-isopropylacrylamide (PNIPAm). The PNIPAm chains may be disposed on the scaffold with a layer thickness of between 0.1 nm to 100 μm. More particularly, the PNIPAm chains may be disposed on the scaffold with a layer thickness of between 0.1 nm to 100 nm.

The cell proliferation apparatus may include a storage tank for storing cell culture medium upstream of the bioreactor, the storage tank being in fluid flow communication with the bioreactor.

The cell proliferation apparatus may include displacement means for displacing cell culture medium from the storage tank to the bioreactor. The displacement means may be a positive displacement pump.

The cell proliferation apparatus may include one or more temperature sensors for monitoring the temperature of any one or more of the cell culture medium, bioreactor, and the scaffold.

The cell proliferation apparatus may include one or more oxygenators for oxygenating any one of the cell culture medium and cells contained in the bioreactor.

The cell proliferation apparatus may include a combined temperature/oxygenator unit.

The cell proliferation apparatus may include a programmable logic controller (PLC) to automate the operating procedures of the system.

The cell proliferation apparatus may include a cell recovery unit in flow communication with, and downstream of, the bioreactor for separation of released cells from the cell culture medium. The cell recovery unit may be a centrifuge, for separation of released cells from the cell culture medium.

An outlet of the cell recovery unit may be connected in fluid flow communication to the cell medium storage tank, to permit the re-use of the cell culture medium. Harvested and separated cells may be entrapped in a cell storage reservoir for later use or may be cryogenically frozen until needed.

The cell proliferation apparatus may include at least one injection/extraction portal on any one, or both sides of the bioreactor, allowing for introduction of biochemicals/chemicals and to allow sampling to be done during operation of the apparatus. This could be for the purposes of introducing chemicals to modulate or change cell behaviour and/or function and/or viability, to monitor cell function and/or viability, or to determine the effect of such chemicals on cell function and/or viability.

In an embodiment in which the grafting technique of solution free radical polymerisation may be used for preparing the scaffold, the solution free radical polymerisation may be accomplished by using any one of redox reagents (e.g. Fe²⁺/H₂O₂), persulphates and thermal initiators (e.g. azo compounds, peroxides, hydroperoxides, peroxide diphosphate, and the like).

When the grafting technique is by means of radiation or photo-induction, both the simultaneous or pre-irradiation methods can be used, where in the former the NIPAm and the scaffold are irradiated in solution simultaneously while with the latter the scaffold is first pre-irradiated prior to being activated (either by heating, or chemical initiation) in the NIPAm solution.

During the solution free radical polymerisation, the homopolymer may be reduced by using multivalent cations, such as Cu²⁺ or Fe²⁺. Preferably the homopolymer may be reduced by using ferrous ammonium sulphate, also known as Mohr's salt.

To enhance the reactivity of the scaffolds polar functional groups may be impregnated/covalently bonded onto the scaffold either prior or during grafting by using any ionization technique selected from any one or more of: radiation techniques such as gamma radiation, plasma radiation, and electron beam radiation; photochemical techniques such as ultra-violet irradiation; ozonation, chemical means such as using persulphate solutions containing multivalent ions, oxyfluorination; or the like. The multivalent ions may, in certain embodiments, be nickel (II) or ceric (IV).

Physical modification techniques may include physical entrapment of PNIPAm chains onto the scaffold surface using swelling/deswelling methods or adsorption techniques.

In the specific case of a hollow fibre membrane bioreactor, oxygenation can occur directly in the bioreactor and temperature control may occur via the inner lumen or extracapillary space (ECS) of the hollow fibres. The temperature release mechanism then occurs directly at the point of cell attachment along the fibres with no drastic change in cell medium temperature as would be necessary in the case of a non-woven or other scaffold. A person skilled in the art may also conceive of other designs that would achieve the same, for example oxygenation exterior to the bioreactor, with internal temperature control of the bioreactor scaffold surfaces eg. by means of liquid circulation within a hollow scaffold.

According to another aspect of the invention, there is provided a method of culturing cells in a non-invasive, continuous manner, the method including the steps of:

providing a bioreactor having included therein a stimulus-responsive three dimensional (3D) scaffold;

seeding cells onto the scaffold;

providing a suitable source of cell culture medium;

allowing the cells to proliferate at a temperature suitable for attachment and proliferation of the cells until a desired cell density has been reached; and

harvesting the cells by changing the surface properties of the stimulus-responsive scaffold from hydrophobic to hydrophilic state, thereby liberating the attached cells.

Examples of cell types could include mammalian primary cells, microbial cells, stem cells, immortalised cell lines, and the like.

The method may include automatically regulating the system parameters with a control system for the proliferation and harvesting of the cells according to a preset programme.

The control system may be regulated through real time measurements of parameters selected from one or more of temperature, pH, flow rates, pressure drop, oxygen consumption, and the like. The input parameters of the system may include metabolic activity for a specific substrate, oxygen consumption, pH, pressure drop and temperature. A programmable logic control (PLC) system is used to automate the operating procedures of the system.

Cells may be allowed to proliferate sufficiently to populate a desired area of the bioreactor scaffold or to a desired density (as determined by oxygen consumption, metabolic activity, pressure drop or other means), the method including the step of either lowering or raising the system temperature to effect a reversible change in hydrophobicity of the scaffold surface, following which the cells are allowed to detach from the scaffold.

The method may include the further step of separating the culture medium and the cell mixture through, for example, centrifugation or any other suitable cell separation/recovery method.

Excess culture medium may be recycled back to a culture medium storage tank for reuse of the cell culture medium.

Harvesting the cells may include lowering the temperature of the oxygen passing through the inner cavity of the hollow fibre scaffold, such that the scaffold surface temperature drops to a temperature at or below the lower critical solution temperature (LCST) of the thermo-responsive material (eg. in the case of PNIPAm, the LCST is 32° C.) to effect cell release. The method therefore allows cells to be selectively released from certain sections of the bioreactor or scaffold. Thus it would be possible to culture different cell types simultaneously in the same bioreactor through selective release of cells from

certain sections and subsequent seeding of a different cell type.

The method may include oxygenation of the cells. Oxygenation of the cells may be performed either via the inner lumen or extracapillary space of the hollow-fibre matrix.

It would thus be possible to set up the system with scaffold sections addressable separately by temperature change, which could also be useful for extracting only a small number of cells from a bioreactor during operation for e.g. DNA/RNA extraction, which would provide valuable information on the state of the cells within the bioreactor. Thus it would enable DNA/RNA monitoring during cell culturing in a 3D environment, which has heretofore to our knowledge not been possible. This ability would be especially useful if the system was applied for applications such as drug screening, where there is frequently a need to know gene expression at different time points during drug administration in vitro, but which is normally not possible, since the normal DNA/RNA extraction techniques require termination of the experiment and destruction of all cells.

Addressable sections in the scaffold may also enable semi-continuous production of cells through cycling release through the addressable sections while allowing sufficient time for repopulation of said sections.

Harvesting the cells may include gradually lowering the temperature of the feed culture medium.

Oxygenation of the cells may be performed via the hollow-fibre matrix, which allows oxygen flowing within the hollow fibre to diffuse out through the fibre into the culture medium. This enables a sufficient supply of oxygen to reach the cells to ensure sufficient cell proliferation. Hollow fibre surface temperature control and oxygenation of cells may be accomplished simultaneously via the inner lumen or either the extracapillary space of the hollow fibres. Oxygen delivery could be enhanced through the use of a synthetic oxygen carrier, such as a perfluorocarbon emulsion or a non-synthetic haemoglobin-based oxygen carrier.

A person skilled in the art will be able to conceive other applications of this system. For example the device can also be used for anchorage independent cell proliferation of suspension cells whereby cells are either trapped in the substrate due to the scaffold pore size (as in the case of hollow fibres) or when the SRP is in an expanded state (as in the case of non-wovens, and gels), and secreted proteins preferentially adsorb onto the SRP coated substrate. This will hence provide selective protein adsorption with retention of either hydrophobic or hydrophilic proteins depending on the LCST or other relevant responsive property of the employed SRP. In the same light, it is thus obvious to use the current system as a combined cell proliferator and selective protein purification device. SRP's that are pH sensitive can also be employed to trap cells and proteins in such a substrate hence also functioning as a cell proliferator and protein purification device.

Further features of the invention will now be described, by way of non-limiting example only, with reference to the accompanying drawing.

### DRAWINGS

In the drawings:

FIG. 1 shows an ATR-FTIR Spectrum of a) pure PP, b) PP-g-PNIPAm (using 10 wt % NIPAm as described in example 1) and c) pure PNIPAm. The presence of the N—H at 3294 cm−1 and 1536 cm−1; and C═O at 1643 cm−1 was detected on the surface of the grafted substrate;

FIG. 2 shows an SEM image of a) pure PP non-woven scaffold, and b) PP-g-PNIPAm non-woven scaffold grafted with 10 wt % NIPAm as per example 1, showing the presence of the grafted layer;

FIG. 3 shows a schematic diagram of a cell proliferation apparatus in accordance with the invention;

FIG. 4 shows an image of cells released from a PNIPAm hollow fiber scaffold after temperature change from 37° C. to 4° C.; and

FIG. 5 shows an image of PP-g-PNIPAm non-woven scaffolds soaked in cell culture media prior to cell inoculation.

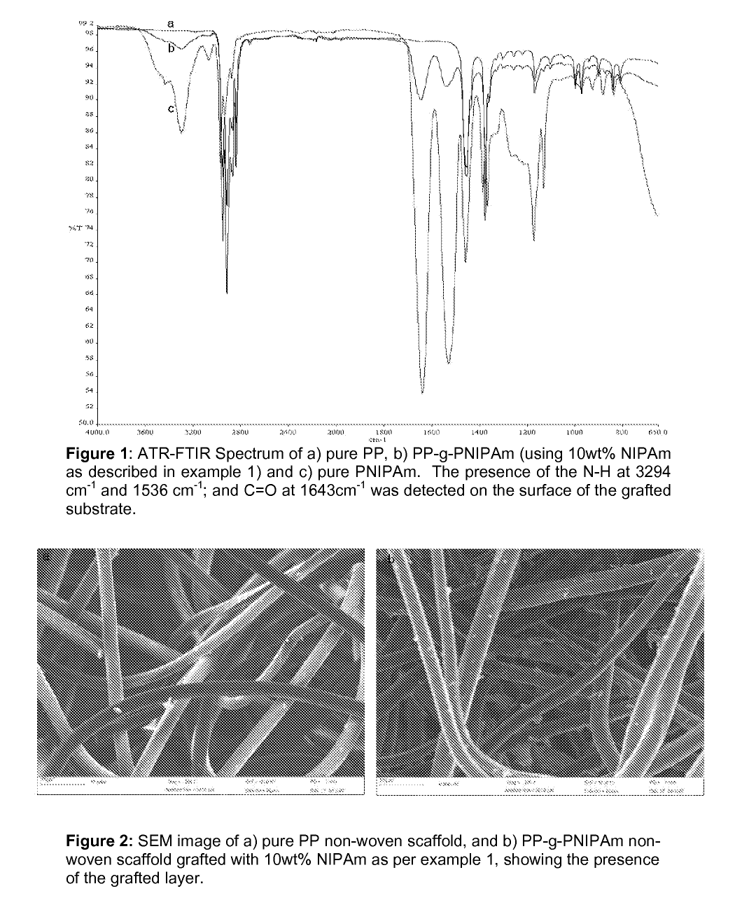

### DETAILED DESCRIPTION OF THE INVENTION

The present invention provides a non-invasive automated cell proliferator. The apparatus includes a stimulus-responsive three dimensional substrate/scaffold whereby proliferated cells are spontaneously released from the system by a change in, or addition of, one or more stimuli. The system has applications in cell and tissue engineering, whereby cell culturing efforts can be scaled up to produce large quantities of viable, in vivo-like 3D cell cultures (or tissue-like constructs), in an easily reproducible and effortless manner. Such an apparatus also finds use in protein and gene expression analysis for genetic engineering.

Poly-N-isopropylacrylamide (PNIPAm) is a polymer which switches reversibly between hydrophobic and hydrophilic states when the temperature crosses its lower critical solution temperature (LCST) of approximately 32° C. This allows cells to attach onto the PNIPAm surface at 37° C. when the surface is hydrophobic while allowing spontaneous release of cells from the hydrophilic surface below the LCST.

It has been found that a major advantage of using PNIPAm for cell culturing is that cells can be harvested non-invasively as intact cell sheets with critical cell surface proteins, growth factor receptors, and cell-to-cell junction proteins remaining intact.

### Example 1

### Solution Free Radical Grafting Method using PP Non-Wovens

Polypropylene (PP) fibres, are needle-punched and thermofused (145° C., 1.5 m/s) into a non-woven mat with density of 130 g/m², and open porosity (40% pores <100 μm, 40% of pores 100-200 μm, and 20% of pores >200 μm). Non-woven mats (6 cm×6 cm×3.21 cm) are washed in ethanol for 1 hour, followed by a water wash prior to drying in the oven at 50° C. Non-woven scaffolds are then placed in a 10 wt % ammonium persulphate (APS) aqueous solution and left to stand for 24 hours at room temperature. Swollen scaffolds are purged with nitrogen gas for 30 minutes, and then placed in an aqueous 10 wt % NIPAm solution pre-bubbled with nitrogen gas for 30 minutes. Grafting is allowed to proceed in a closed vessel at 70° C. for 24 hours. Grafted fibres scaffolds are then washed in cold deionised water for 3 days. Purity of the grafted mats is verified by monitoring the water washes using UV-VIS spectroscopy at 190-400 nm and by observing the turbidity of the washes at 45° C. and finally dried in an oven at 50° C. The grafted non-wovens are autoclaved at 120° C. for 15 minutes prior to cell culturing (see example 7). Grafting is confirmed by Attenuated Total Reflectance—Fourier Transform Infrared Spectrophotometry (ATR-FTIR) scanning and scanning electron microscopy (SEM).

### Example 2

### Solution Free Radical Grafting Method 2 using PP Non-Wovens

To increase graft yield, example 1 can be repeated, with the addition of 0.25 wt % ammonium iron (II) sulphate hexahydrate (Mohr's salt) to the NIPAm solution prior to grafting.

### Example 3

### Solution Free Radical Grafting by Method 3

PP non-wovens (6 cm×6 cm×3.21 cm) are washed as described previously. Scaffolds are placed in a 10 wt % APS aqueous solution and heated at 80° C. for 3 hours, followed by thorough washing in deionised water. The treated scaffolds are then placed in an aqueous 10 wt % NIPAm solution containing 0.002 M ammonium cerium (IV) nitrate and 0.04 M nitric acid pre-bubbled with nitrogen gas for 30 minutes. Grafting is allowed to proceed in an oven at 50° C. for 24 hours. Grafted scaffolds are then washed for 3 days and dried as described previously. Grafting is confirmed by ATR-FTI R.

### Example 4

### Solution Free Radical Grafting using PP Hollow-Fibre Cartridge

Cellmax PP hollow fibre cartridge (pore size: 0.5 μm, outer diameter: 630 μm) is filled with a 10 wt % APS aqueous solution and left to stand for 24 hours at room temperature. The APS solution is then drained, and replaced with an aqueous 10 wt % NIPAm solution pre-bubbled with nitrogen gas for 30 minutes. The cartridge is then placed in a water bath at 70° C. for 5 hours. The grafted cartridge is then perfused with cold deionised water for 2 days using a peristaltic pump. Purity of the grafted hollow fibres is verified by monitoring the water washes using UV-VIS spectroscopy at 190-400 nm and by checking the turbidity of the washes at 45° C. The grafted cartridge is autoclaved at 120° C. for 15 minutes prior to cell culturing (see example 6 below).

### Example 5

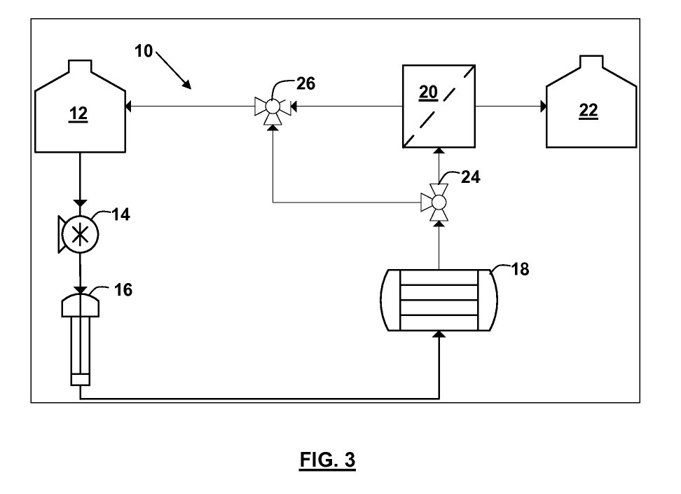

Referring now to FIG. 3, the components of a cell proliferation apparatus 10 in accordance with the invention are described. Furthermore, the operation of the cell proliferation apparatus 10 is described. Buffered cell culture media contained in a reservoir 12 is pumped by means of a positive displacement pump 14 to the temperature and/or oxygenator unit 16, which is used to control the temperature and/or oxygenate cells contained in a cell-seeded bioreactor 18. The bioreactor contains a stimulus-responsive three-dimensional (3D) cell scaffold.

A temperature-responsive substrate in the bioreactor 18 will be triggered by a change in temperature to release cells, which are attached to the scaffold. The cells are then recovered via a cell separator 20 and are stored in a storage facility 22.

During cell growth the cell containing media from the reservoir is passed through two three-way valves 24 and 26, back to the reservoir 12.

In the specific embodiment of a hollow fibre membrane bioreactor 16, oxygenation can occur directly in the bioreactor while the temperature of the media is accurately controlled to maintain cell growth. Oxygenation then takes place via the inner lumen of the hollow fibres. In this embodiment, the temperature release mechanism is initiated directly at the point of cell attachment along the fibres with no drastic change in cell media temperature as would be necessary in the embodiment of a nonwoven substrate.

Persons of ordinary skill in the art will appreciate that different configurations and components of the cell proliferation apparatus 10 may yield similar results, and this invention is therefore not limited to the above example.

### Example 6

### Culturing of Hep3G Cells in the Cellmax PP-g-PNIPAm Hollow Fibre Bioreactor

A hollow fibre scaffold/cartridge is grafted with NIPAm as described in example 4. Hep3B hepatocytes are cultured in the lumen of the grafted cartridge. The cell culture media consist of EMEM (with L-glutamine) supplemented with 10% FBS and 1% Pen/Strep antibiotics. Prior to cell inoculation, the PNIPAm grafted polypropylene cartridge is pre-cultured with media for 1 day at 37° C. in an incubator. Cells are then inoculated in the lumen at a cell density of 2×10⁶ in an incubator at 37° C. with 5% CO₂, 20% O₂ and 75% N₂. The cells are allowed to attach statically for 1 hour with a 30 minute rotation to spread cell attachment throughout the fibers. The media is continuously perfused through the extra capillary space (ECS) and is changed once a day within the 2 day culturing period. For cell release on the second day, media at a temperature of about 4° C. is perfused through the ECS for 30 minutes while media pre-warmed to 37° C. is passed through the lumen. All released cells are then collected in a separate reservoir for further analysis.

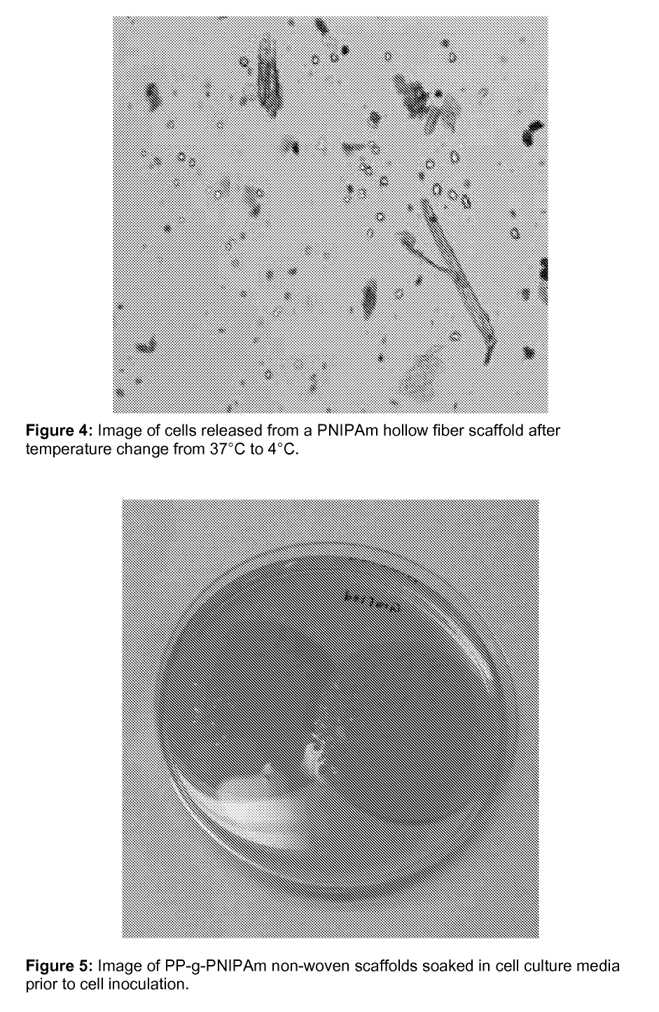

FIG. 4 illustrates the morphology of the released cells. It can be seen that many particles and cell sheets were released. It has been found that the indirect temperature release method allows for effective cell release while maintaining the recovered cells in an optimum temperature of 37° C.

### Example 7

### Culturing of Hep3G Cells onto PP-g-PNIPAm Non-Woven Scaffold

PNIPAm grafted polypropylene (PP) nonwoven having a diameter of 4 cm are grafted and sterilised as described in example 1. Prior to cell inoculation grafted disks are pre-cultured with media for 1 day under similar conditions as described in example 7. Cell inoculation is then undertaken by seeding 3×10⁵ cells/ml to a small area of the non-woven disk. Cells are added drop-wise and allowed to attach for 1 hour. Cells are then cultured for 2 days with 1 day media change in static culture in an incubator at 37° C. To initiate cell release, media is replaced with chilled media (4° C.) and released cells are then collected for further analysis. The presence of many particles and cell sheets can be observed. This example illustrates the mechanism of cell release from a non-woven scaffold by inducing a change in media temperature.

The inventors are of the opinion that they have invented an automated cell proliferation apparatus and method, which has numerous advantages over conventional cell culturing techniques. Such advantages include the fact that the invention represents a useful automated cell proliferation apparatus incorporating a thermo-responsive scaffold for high-throughput cell culturing, without any invasive techniques being required from a user. The apparatus of the invention is thus suitable for high through-put cell culturing and reduces the time-consuming efforts required for conventional cell culturing techniques. It also significantly reduces the risk of contamination. As such, the apparatus comprises a 3D thermo-responsive scaffold capable of releasing cells without requiring enzymes such as trypsin or other aggressive cell removal methods. The apparatus has a 3D thermo-responsive scaffold which has the potential to produce cell cultures with improved maintenance of cell differentiation and function compared to monolayer cultures. Additionally, the apparatus provides a gentle cell release trigger such that cells are not exposed to a drastic temperature change in cell culture medium.

In addition, in certain embodiments, the non-invasive cell proliferation apparatus is conveniently provided with injection/extraction portals on either or both sides of the bioreactor, allowing for introduction of biochemicals or chemicals and sampling to be done during operation of the apparatus. This could be for the purposes of introducing chemicals to modulate or change cell behaviour and/or function and/or viability, to monitor cell function and/or viability, or to determine the effect of such chemicals on cell function and/or viability.

The complete bioreactor system, including the bioreactor housing, the cell scaffold, piping, the reservoir, and the like, can be constructed from sterilizable plastic components. In addition, the apparatus of the invention is a compact, modular, user-friendly and cost-effective apparatus for cell proliferation and harvesting.

1. A cell proliferation apparatus for the automated culturing of cells, the proliferation apparatus including a bioreactor having contained therein a stimulus-responsive three dimensional (3D) cell scaffold.

2. A cell proliferation apparatus as claimed in claim 1, in which the stimulus-responsive three-dimensional (3D) cell scaffold reversibly changes its surface properties between hydrophilic and hydrophobic states.

3. A cell proliferation apparatus as claimed in any one of claims 1 and 2, in which the scaffold material is defined by a matrix selected from any one or more of fibres, semi-permeable or non-permeable hollow fibres, hydrogels, particles and monolithic porous scaffolds made from either polymers or ceramics.

4. A cell proliferation apparatus as claimed in any one of claims 1 to 3, in which the scaffold comprises a semi-permeable hollow fibre matrix.

5. A cell proliferation apparatus as claimed in any one of claims 1 to 3, in which the scaffold is selected from any one of polystyrene, polypropylene, polyethylene, polyesters, polyamides, and natural polymers.

6. A cell proliferation apparatus as claimed in any one of claims 1 to 5, in which the scaffold is modified with a surface layer of thermo-responsive polymer by grafting.

7. A cell proliferation apparatus as claimed in claim 6, in which the grafting technique is selected from any one or more of: solution free radical polymerisation; gamma radiation; plasma radiation; electron beam radiation; and ultra-violet radiation.

8. A cell proliferation apparatus as claimed in any one of claims 1 to 5, in which the scaffold is modified with a surface layer of thermo-responsive polymer by physical adsorption or attachment techniques.

9. A cell proliferation apparatus as claimed in any one of claims 1 to 8, in which the thermo-responsive polymer is selected from any one or more of poly N-substituted acrylamide, polyethylene-oxide, and their respective copolymers.

10. A cell proliferation apparatus as claimed in claim 9, in which the thermo-responsive polymer is poly-N-isopropylacrylamide (PNIPAm).

11. A cell proliferation apparatus as claimed in claim 10, in which the PNIPAm chains are disposed on the scaffold with a layer thickness of between 0.1 nm to 100 μm.

12. A cell proliferation apparatus as claimed in claim 11, in which the PNIPAm chains are disposed on the scaffold with a layer thickness of between 0.1 nm to 100 nm.

13. A cell proliferation apparatus as claimed in any one of claims 1 to 12, which includes a storage tank for storing cell culture medium upstream of the bioreactor, the storage tank being in fluid flow communication with the bioreactor.

14. A cell proliferation apparatus as claimed in any one of claims 1 to 13, which includes displacement means for displacing cell culture medium from the storage tank to the bioreactor.

15. A cell proliferation apparatus as claimed in claim 14, in which the displacement means is a positive displacement pump.

16. A cell proliferation apparatus as claimed in any one of claims 1 to 15, which includes one or more temperature sensors for monitoring the temperature of any one or more of the cell culture medium, bioreactor, and the scaffold.

17. A cell proliferation apparatus as claimed in any one of claims 1 to 16, which includes one or more oxygenators for oxygenating the cell culture medium or the cells contained in the bioreactor.

18. A cell proliferation apparatus as claimed in any one of claims 1 to 15, which includes a combined temperature/oxygenator unit.

19. A cell proliferation apparatus as claimed in any one of claims 1 to 18, which includes a programmable logic controller (PLC) to automate the operating procedures of the system.

20. A cell proliferation apparatus as claimed in any one of claims 12 to 19, which includes a cell recovery unit in flow communication with, and downstream of, the bioreactor for separation of released cells from the cell culture medium.

21. A cell proliferation apparatus as claimed in claim 20 in which an outlet of the cell recovery unit is connected in fluid flow communication to the cell medium storage tank, to permit the re-use of the cell culture medium.

22. A cell proliferation apparatus as claimed in any one of claims 1 to 21 which includes at least one injection/extraction portal on any one, or both sides of the bioreactor, allowing for introduction of biochemicals or chemicals and allowing for sampling to be done during operation of the apparatus.

23. A cell proliferation apparatus as claimed in claim 7, in which the grafting technique of solution free radical polymerisation is used, the solution free radical polymerisation being accomplished using any one of redox reagents, persulphates and thermal initiators.

24. A cell proliferation apparatus as claimed in claim 23, in which homopolymer formation is reduced by using multivalent cations.

25. A cell proliferation apparatus as claimed in claim 24, in which homopolymer formation is reduced by using ferrous ammonium sulphate.

26. A method of culturing cells in a non-invasive, continuous manner, the method including the steps of:
providing a bioreactor having included therein a stimulus-responsive three dimensional (3D) scaffold;
seeding cells onto the scaffold;
providing a suitable source of cell culture medium;
allowing the cells to proliferate at a temperature suitable for attachment and proliferation of the cells until a desired cell density has been reached; and
harvesting the cells by changing the surface properties of the stimulus-responsive scaffold from hydrophobic to hydrophilic state, thereby liberating the attached cells.

27. A method as claimed in claim 26, in which a control system automatically regulates the system parameters for the proliferation and harvesting of the cells according to a preset programme.

28. A method as claimed in claim 27, in which the control system is regulated through real time measurements of parameters selected from one or more of temperature, pH, flow rates, pressure drop and oxygen consumption.

29. A method as claimed in claim 28, which includes input parameters to the control systems selected from any one or more of metabolic activity for a specific substrate, oxygen consumption, pH, pressure drop and temperature.

30. A method as claimed in claim 26, wherein cells are allowed to proliferate sufficiently to populate a desired area of the bioreactor scaffold or to a desired density, the method including the step of either lowering or raising the system temperature to effect a reversible change in hydrophobicity of the scaffold surface, following which the cells are allowed to detach from the scaffold.

31. A method as claimed in claim 30, in which the cells are selectively released from certain sections of the bioreactor or scaffold.

32. A method as claimed in claim 26, which includes the further step of separating the culture medium and the cell mixture.

33. A method as claimed in claim 32 in which excess culture medium is recycled back to a culture medium storage tank for reuse of the cell culture medium.

34. A method as claimed in claim 26, in which harvesting the cells includes lowering the temperature of the oxygen passing through the inner cavity of the hollow fibre scaffold, such that the scaffold surface temperature drops to a temperature at or below the lower critical solution temperature (LCST) to effect cell release.

35. A method as claimed in claim 26, in which harvesting the cells includes gradually lowering the temperature of the feed culture medium below the LCST.

36. A method as claimed in claim 26, which includes oxygenation of the cells.

37. A method as claimed in claim 36, in which oxygenation of the cells is performed either via the inner lumen or extracapillary space of the hollow-fibre matrix.

38. A cell proliferation apparatus as claimed in claim 1, substantially as herein described and illustrated.

39. A method as claimed in claim 26, substantially as herein described and illustrated.

40. A cell proliferation apparatus and a new method, substantially as herein described.
